(19) United States
(12) Patent Application Publication
(10) Pub. No.: US 2005/0201567 A1
(43) Pub. Date: Sep. 15, 2005

(54) Tunable exhaust system

(76) Inventors: Alan L. Browne, Grosse Pointe, MI (US); Donald J. Nefske, Troy, MI (US); Shung H. Sung, Troy, MI (US)

Correspondence Address: General Motors Corporation, Legal Staff;Mail Code 482-CCS-B21, 300 Renaissance Center, PO Box 300, Detroit, MI, 48265, US

(21) Appl. No.: 11/077,672

(22) Filed: Mar. 10, 2005

Related U.S. Application Data
(60) Provisional application No. 60/552,794, filed on Mar. 12, 2004.

(51) Int. Cl. A61F 11/06; G10K 11/16; H03B 29/00

(52) U.S. Cl. 381/71.5; 181/224

(57) ABSTRACT

An exhaust system includes an exhaust conduit; a piezoelectric patch secured to the exhaust conduit; a sensor in operative communication with the exhaust conduit; and a controller in operative communication with the piezoelectric patch and the sensor, wherein the controller is configured to provide a signal to the piezoelectric patch and modify a sound emitted from the exhaust system. A method includes producing an exhaust gas in an exhaust conduit of an exhaust system; measuring an acoustic property of the exhaust gas, an acoustic property of the exhaust conduit, or a combination comprising at least one of the foregoing acoustic properties; applying an electrical signal to a piezoelectric patch, wherein the piezoelectric patch is secured to the exhaust conduit; and deforming the piezoelectric patch mechanically to modulate a sound emitted from the exhaust system.

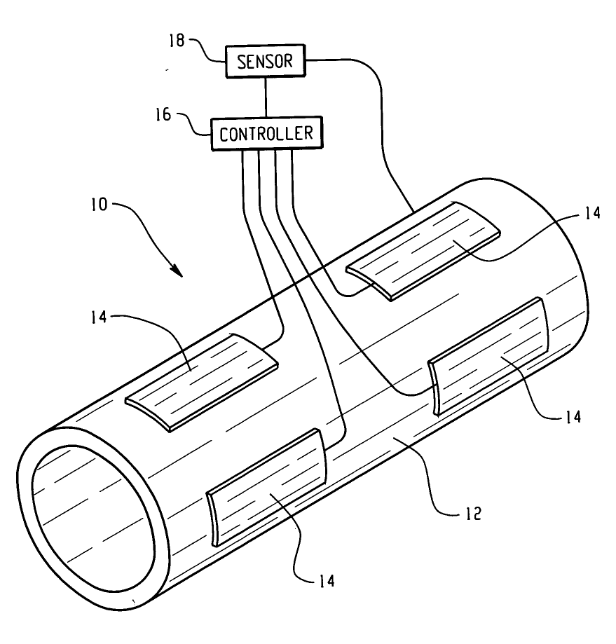

### CROSS REFERENCE TO RELATED APPLICATIONS

The present application relates to, and claims priority to, U.S. Provisional Patent Application No. 60/552,794, which was filed on Mar. 12, 2004 and is incorporated herein in its entirety.

### BACKGROUND

This disclosure relates to exhaust systems and, more particularly, to tunable exhaust systems for motor vehicles.

Current exhaust systems are engineered to emit a distinctive sound that is desired for a particular motor vehicle. Obtaining the desired sound requires careful selection and control over the numerous components associated with the exhaust system. These include the exhaust diameter, exhaust length, muffler design, resonator design, catalytic converter design, manifold design, flow pattern of the exhaust gases through the exhaust system, and hanging configurations, among others.

Many motor vehicle users turn to aftermarket exhaust systems for a different sound than that emitted from the originally installed exhaust system. For example, some motor vehicle users prefer relatively loud grumbling sounds, which are suggestive of engine power. However, another user of the same motor vehicle may prefer a quieter sound emitted from the exhaust system. Furthermore, there are certain locations (e.g., near hospitals, schools, libraries, places of worship, and the like), and/or times (e.g., during the hours when people may be sleeping) when loud sound is discouraged or even prohibited.

Accordingly, new and improved exhaust systems that can be variably tuned to the preferences of the motor vehicle user and/or situation are needed. It would be particularly advantageous if these systems did not adversely affect the performance of the motor vehicle, such as by decreasing horsepower, increasing pollutant emissions, and the like.

### BRIEF SUMMARY

An exhaust system includes an exhaust conduit; a piezoelectric patch secured to the exhaust conduit; a sensor in operative communication with the exhaust conduit; and a controller in operative communication with the piezoelectric patch and the sensor, wherein the controller is configured to provide a signal to the piezoelectric patch and modify a sound emitted from the exhaust system.

In another aspect, the exhaust system includes an exhaust conduit; a piezoelectric patch secured to the exhaust conduit; a sensor located upstream of the piezoelectric patch and in operative communication with the exhaust conduit, wherein the upstream sensor is configured to measure information comprising an acoustic property of the exhaust conduit, an acoustic property of an exhaust gas in the exhaust conduit, or a combination comprising one of the foregoing acoustic properties; and a controller in operative communication with the piezoelectric patch and the upstream sensor, wherein the controller is adapted to receive measured information from the upstream sensor and is operable to extract an amplitude, frequency and/or phase of the measured information and to selectively apply a predictive voltage to the piezoelectric patch to effect a transient mechanical deformation of the piezoelectric patch, wherein the transient mechanical deformation of the piezoelectric patch results in a transient localized distortion of a shape of the exhaust conduit, wherein the transient localized distortion of the shape of the exhaust conduit results in modulation of an emitted sound from the exhaust system.

A method includes producing an exhaust gas in an exhaust conduit of an exhaust system; measuring an acoustic property of the exhaust gas, an acoustic property of the exhaust conduit, or a combination comprising at least one of the foregoing acoustic properties; applying an electrical signal to a piezoelectric patch, wherein the piezoelectric patch is secured to the exhaust conduit; and deforming the piezoelectric patch mechanically to modulate a sound emitted from the exhaust system.

The above described and other features are exemplified by the following FIGURE and detailed description.

### BRIEF DESCRIPTION OF THE DRAWING

Referring now to the FIGURE, which is an exemplary embodiment and wherein like elements are numbered alike:

The FIGURE is a schematic representation of a section of an exhaust system.

### DETAILED DESCRIPTION

Disclosed herein are exhaust systems and methods of use in any application wherein control of a sound emitted from an exhaust system is desired. In contrast to the prior art, the exhaust systems and methods disclosed herein are advantageously based on piezoelectric materials. As used herein, the term “piezoelectric” generally refers to a material that mechanically deforms when an electrical signal is applied or, conversely, generates an electrical signal when mechanically deformed.

Also, as used herein, the terms “first”, “second”, and the like do not denote any order or importance, but rather are used to distinguish one element from another, and the terms “the”, “a”, and “an” do not denote a limitation of quantity, but rather denote the presence of at least one of the referenced items. Furthermore, all ranges disclosed herein are inclusive of the endpoints and independently combinable.

Referring now to The FIGURE, a portion of an exemplary exhaust system 10 is shown. The exhaust system 10 includes an exhaust conduit 12 and a piezoelectric patch 14 that is secured to the exhaust conduit 12. As used herein, the term “conduit” refers to any device in which an exhaust may flow from a first location (e.g., from the engine cylinder) to a second location (e.g., downstream of the engine cylinder) and may be of any size or shape. The piezoelectric patch 14 is advantageously able to modulate the sound associated with the emitted exhaust by mechanically acting on the exhaust conduit 12.

The exhaust system 10 further includes a controller 16 in operative communication with the piezoelectric patch 14. The controller 16 is operable to selectively apply an electrical signal to the piezoelectric patch 14 to effect a transient mechanical deformation of the patch 14, which enables the exhaust conduit 12 to undergo a transient localized distortion in its shape.

The exhaust system 10 further includes a sensor 18 in operative communication with the controller 16. The sensor 18 is configured to provide information to the controller 16 for selectively applying the electrical signal to effect the transient mechanical deformation of the piezoelectric patch 14. In one embodiment, the sensor 18 is configured to measure the acoustically induced (i.e., sound-pressure) surface vibration of the exhaust conduit 12 and generate a representative electrical signal of this vibration. In another embodiment, the sensor 18 is configured to measure the acoustical energy (i.e., sound-power) of the exhaust gas flow in the exhaust conduit 12 and generate a representative electrical signal of this acoustical energy. The controller 16 receives the information (e.g., in the form of the electrical signal); extracts the spectral content, amplitude and/or phase; and applies an appropriate electrical signal to the piezoelectric patch 14. The piezoelectric patch 14 accordingly undergoes a transient mechanical deformation, resulting in the exhaust conduit 12 experiencing a transient localized distortion in shape. The sensor 18 may be a vibration sensor that is surface mounted (not shown) or otherwise disposed in operative communication with the exhaust conduit to measure the conduit surface vibration, and/or the sensor 18 may be an acoustic sensor or microphone within the exhaust conduit (not shown) or otherwise disposed in operative communication with the exhaust gas flow in the conduit to measure the acoustical energy of the exhaust gas.

While The FIGURE illustrates four equal sized piezoelectric patches 14 secured to the exhaust conduit 12, the size, shape, location, and number of piezoelectric patches 14 will depend on the specific level or extent of sound frequency and/or magnitude modulation desired, and will be apparent to those skilled in the art in view of this disclosure. For example, if a finite frequency and/or magnitude modulation window is desired, then the exhaust system 10 may comprise fewer, smaller, and/or more spread out piezoelectric patches 14 than an exhaust system 10 wherein a larger frequency and/or magnitude modulation window is desired.

In operation of the exhaust system 10, the motor vehicle engine produces an exhaust with a sound, or exhaust-gas acoustical energy, that varies according to the engine revolution speed and load. The sensor 18 measures the acoustically induced vibration of the exhaust conduit 12 and/or the acoustical energy of the exhaust gas flow in the exhaust conduit 12. From this measurement, the sensor 18 generates an electrical signal representative of the measured vibration and/or energy, and it provides this information to the controller 16, which then extracts the spectral content, amplitude and/or phase. Generally the frequency of the exhaust sound is about 10 Hertz (Hz) to about 10 kilohertz (kHz); and the amplitude of the exhaust sound is about 50 decibels (dB) to about 115 dB. The controller 16, based on the information provided by the sensor 18 and the selected sound desired by the motor vehicle user, applies an electrical signal to the piezoelectric patch 14 to effect the transient mechanical deformation of the piezoelectric patch 14, which enables the exhaust conduit 12 to undergo a transient localized distortion in its shape, and thereby modulate the exhaust gas acoustical energy and the emitted sound. Owing to the fact that this process (i.e., sensing, extracting, applying electrical signal, and deforming the patch) is a continuous loop, the piezoelectric patch 14 does not experience a discrete static deformation, but instead vibrates in a transient manner to alter the exhaust gas acoustical energy.

If the sound of the exhaust is above the selected sound level (i.e., amplitude) desired by the motor vehicle user, then the controller 16 applies the electrical signal such that the piezoelectric patch 14 destructively interferes with, and therefore dampens, the exhaust sound. Alternatively, if the sound of the exhaust is below the selected sound level desired by the motor vehicle user, then the controller 16 applies the electrical signal such that the piezoelectric patch 14 constructively interferes with, and therefore heightens, the exhaust sound.

Furthermore, if the sound of the exhaust is of a different spectral character than desired by the motor vehicle user, then the controller 16 applies the electrical signal such that the piezoelectric patch 14 alters the spectral character of the conduit vibration and thereby the spectral character of the exhaust sound.

In one embodiment, the sensor 18 is a vibration sensor, such as a different piezoelectric patch, that is secured to the exhaust conduit 12 upstream, downstream, or proximate to the piezoelectric patch 14. The sensor 18 generates an electrical signal, representative of the measured conduit vibration, which is sent to the controller 16. Based on the spectral content, amplitude and/or phase of the electrical signal and the selected sound level and/or spectral character desired by the motor vehicle user, the controller 16 applies an electrical signal to the piezoelectric patch 14 to tune the sound of the exhaust. If the sensor 18 is upstream of the piezoelectric patch 14, then the electrical signal applied by the controller 16 to tune the sound of the exhaust is termed a predictive electrical signal. If, however, the sensor 18 is downstream of the piezoelectric patch 14, then the electrical signal applied by the controller 16 to tune the sound of the exhaust is termed a corrective electrical signal.

In another embodiment, the sensor 18 is an acoustical sensor, such as a microphone, that is positioned inside the exhaust conduit upstream, downstream, or proximate to piezoelectric patch 14. The sensor 18 generates an electrical signal representative of the measured acoustical energy that is sent to the controller 16. Based on the spectral content, amplitude and/or phase of this signal and the elected sound level and/or spectral character desired by the motor vehicle user, the controller 16 applies the electrical signal to the piezoelectric patch 14 to tune the sound of the exhaust. If the sensor 18 is upstream of the piezoelectric patch 14, then the predictive electrical signal is applied by the controller 16; and if the sensor 18 is downstream of the piezoelectric patch 14, then the corrective electrical signal is applied by the controller 16.

In still another embodiment, the sensor 18 further comprises a acoustical sensor and/or a vibration sensor that is positioned upstream of the piezoelectric patch 14 and independently an acoustical sensor and/or vibration sensor that is positioned either downstream or at the same location as the piezoelectric patch 14. Not only is any signal from the exhaust conduit 12 that is picked up by the upstream sensor processed by the controller 16, but any signal from the exhaust conduit 12 that is picked up by the downstream sensor is also processed by the controller 16. In this manner, the controller 16 can more accurately tune the sound of the exhaust to the selected sound level and/or spectral character desired by the motor vehicle user.

The desired sound level and/or sound spectrum may be manually selected by the motor vehicle user during operation of the motor vehicle, or may be automatically set based on the time and location of vehicle operation.

The choice of material for the piezoelectric patch 14 will depend on the conditions to which it will be exposed. For example, a material with greater temperature stability will be required as the patch 14 is secured to the exhaust conduit 12 closer to the point of discharge of the exhaust from the engine into the exhaust system 10. As the distance from the engine increases, the temperature stability of the piezoelectric patch 14 becomes less of a concern.

An exemplary piezoelectric patch includes a layer of a piezoelectric material sandwiched between electrodes that are encapsulated by a protective layer. During fabrication, the structure is held together with an adhesive, such as a polyimide tape, and placed in an autoclave for processing through a prescribed temperature-and-pressure cycle.

Preferably, a piezoelectric material is disposed on strips of a flexible metal or ceramic sheet. The strips can be unimorph or bimorph. Preferably, the strips are bimorph, because bimorphs generally exhibit more displacement than unimorphs.

One type of unimorph is a structure composed of a single piezoelectric element externally bonded to a flexible metal foil or strip, which is stimulated by the piezoelectric element when activated with a changing electrical charge and results in an axial buckling or deflection as it opposes the movement of the piezoelectric element. The actuator movement for a unimorph can be by contraction or expansion.

In contrast to the unimorph piezoelectric device, a bimorph device includes an intermediate flexible metal foil sandwiched between two piezoelectric elements. Bimorphs exhibit more displacement than unimorphs because under the applied electrical charge one ceramic element will contract while the other expands.

Suitable piezoelectric materials include, but are not intended to be limited to, inorganic compounds, organic compounds, and metals. With regard to organic materials, all of the polymeric materials with non-centrosymmetric structure and large dipole moment group(s) on the main chain or on the side-chain, or on both chains within the molecules, can be used as suitable candidates for the piezoelectric film. Exemplary polymers include, for example, but are not limited to, poly(sodium 4-styrenesulfonate), poly (poly(vinylamine)backbone azo chromophore), and their derivatives; polyfluorocarbons, including polyvinylidenefluoride, its co-polymer vinylidene fluoride (“VDF”), co-trifluoroethylene, and their derivatives; polychlorocarbons, including poly(vinyl chloride), polyvinylidene chloride, and their derivatives; polyacrylonitriles, and their derivatives; polycarboxylic acids, including poly(methacrylic acid), and their derivatives; polyureas, and their derivatives; polyurethanes, and their derivatives; bio-molecules such as poly-L-lactic acids and their derivatives, and cell membrane proteins, as well as phosphate bio-molecules such as phosphodilipids; polyanilines and their derivatives, and all of the derivatives of tetramines; polyamides including aromatic polyamides and polyimides, including Kapton and polyetherimide, and their derivatives; all of the membrane polymers; poly(N-vinyl pyrrolidone) (PVP) homopolymer , and its derivatives, and random PVP-co-vinyl acetate copolymers; and all of the aromatic polymers with dipole moment groups in the main-chain or side-chains, or in both the main-chain and the side-chains, and mixtures thereof.

Piezoelectric materials can also comprise metals, such as lead, antimony, manganese, tantalum, zirconium, niobium, lanthanum, platinum, palladium, nickel, tungsten, aluminum, strontium, titanium, barium, calcium, chromium, silver, iron, silicon, copper, alloys comprising at least one of the foregoing metals, and oxides comprising at least one of the foregoing metals. Suitable metal oxides include SiO₂, Al₂O₃, ZrO₂, TiO₂, SrTiO₃, PbTiO₃, BaTiO₃, FeO₃, Fe₃O₄, ZnO, and mixtures thereof. Other piezoelectric materials include Group VIA and IIB compounds, such as CdSe, CdS, GaAs, AgCaSe₂, ZnSe, GaP, InP, ZnS, and mixtures thereof. Specific desirable piezoelectric materials are polyvinylidene fluoride, lead zirconate titanate (PZT), and barium titanate.

Generally, electrodes suitable for use may be of any shape and material provided that they are able to supply a suitable electrical charge to, or receive a suitable electrical charge from, the piezoelectric material. The electrical charge may be either constant or varying over time. In one embodiment, the electrodes adhere to a surface of the piezoelectric. Electrodes adhering to the piezoelectric are preferably compliant and conform to the changing shape of the piezoelectric. Correspondingly, the present disclosure may include compliant electrodes that conform to the shape of the piezoelectric to which they are attached. The electrodes may be only applied to a portion of a piezoelectric and define an active area according to their geometry. Various types of electrodes suitable for use with the present disclosure include structured electrodes comprising metal traces and charge distribution layers, textured electrodes comprising varying out of plane dimensions, conductive greases such as carbon greases or silver greases, colloidal suspensions, high aspect ratio conductive materials such as carbon fibrils and carbon nanotubes, and mixtures of ionically conductive materials.

Other suitable materials used in an electrode include graphite, carbon black, colloidal suspensions, thin metals including silver and gold, silver filled and carbon filled gels and polymers, and ionically or electronically conductive polymers. It is understood that certain electrode materials may work well with particular polymers and may not work as well for others. By way of example, carbon fibrils work well with acrylic elastomer polymers while not as well with silicone polymers.

Advantageously, the above noted exhaust systems provide a means of controllably tuning the sound emitted from an exhaust to a desired level. In addition to providing tunability, it should be recognized by those skilled in the art that because these systems do not require any changes to internal components of an exhaust system, they can control sound without adversely affecting the performance of the motor vehicle.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

1. An exhaust system, comprising:
an exhaust conduit;
a piezoelectric patch secured to the exhaust conduit;
a sensor in operative communication with the exhaust conduit; and
a controller in operative communication with the piezoelectric patch and the sensor, wherein the controller is configured to provide an electrical signal to the piezoelectric patch and modify a sound emitted from the exhaust system.

2. The exhaust system of claim 1, wherein the sensor is configured to measure an acoustic property of the exhaust conduit, an acoustic property of an exhaust gas in the exhaust conduit, or a combination comprising one of the foregoing acoustic properties.

3. The exhaust system of claim 1, wherein the sensor comprises a microphone, an other piezoelectric patch, or a combination comprising at least one of the foregoing, located upstream of the piezoelectric patch.

4. The exhaust system of claim 1, wherein the sensor comprises a microphone, an other piezoelectric patch, or a combination comprising at least one of the foregoing, located downstream of the piezoelectric patch.

5. The exhaust stream of claim 3, wherein the sensor further comprises a microphone, other piezoelectric patch, or a combination comprising at least one of the foregoing, located upstream of the piezoelectric patch.

6. The exhaust system of claim 1, wherein the sensor is mounted on an outer surface of the exhaust conduit.

7. The exhaust system of claim 1, wherein the piezoelectric patch comprises lead zirconate titanate.

8. The exhaust system of claim 1, wherein the modulation occurs by wave interference.

9. An exhaust system, comprising:
an exhaust conduit;
a piezoelectric patch secured to the exhaust conduit;
a sensor located upstream of the piezoelectric patch and in operative communication with the exhaust conduit, wherein the upstream sensor is configured to measure information comprising an acoustic property of the exhaust conduit, an acoustic property of an exhaust gas in the exhaust conduit, or a combination comprising one of the foregoing acoustic properties upstream of the piezoelectric patch; and
a controller in operative communication with the piezoelectric patch and the upstream sensor, wherein the controller is adapted to receive upstream measured information from the upstream sensor and is operable to extract an amplitude, frequency and/or phase of the upstream measured information and to selectively apply a predictive voltage to the piezoelectric patch to effect a transient mechanical deformation of the piezoelectric patch, wherein the transient mechanical deformation of the piezoelectric patch results in a transient localized distortion of a shape of the exhaust conduit, wherein the transient localized distortion of the shape of the exhaust conduit results in modulation of an emitted sound from the exhaust system.

10. The exhaust system of claim 9, wherein the upstream sensor is a microphone, an other piezoelectric patch, or a combination comprising at least one of the foregoing.

11. The exhaust system of claim 9, further comprising a sensor located downstream of the piezoelectric patch and in operative communication with the exhaust conduit, wherein the upstream sensor is configured to measure information comprising an acoustic property of the exhaust conduit, an acoustic property of an exhaust gas in the exhaust conduit, or a combination comprising one of the foregoing acoustic properties downstream of the piezoelectric patch.

12. The exhaust system of claim 11, wherein the downstream sensor is a microphone, an other piezoelectric patch, or a combination comprising at least one of the foregoing.

13. The exhaust system of claim 11, wherein the controller is further in operative communication with the downstream sensor, and is further adapted to receive downstream measured information from the downstream sensor, and is further operable to extract the amplitude, frequency and/or phase of the downstream measured information and to further selectively apply a corrective voltage to the piezoelectric patch to effect the transient mechanical deformation of the piezoelectric patch.

14. The exhaust system of claim 9, wherein the piezoelectric patch comprises lead zirconate titanate.

15. The exhaust system of claim 9, wherein the modulation occurs by wave interference.

16. A method, comprising:
producing an exhaust gas in an exhaust conduit of an exhaust system;
measuring an acoustic property of the exhaust gas, an acoustic property of the exhaust conduit, or a combination comprising at least one of the foregoing acoustic properties;
applying an electrical signal to a piezoelectric patch, wherein the piezoelectric patch is secured to the exhaust conduit; and
deforming the piezoelectric patch mechanically to modulate a sound emitted from the exhaust system.

17. The method of claim 16, further comprising repeating, in sequence, the measuring, applying, and deforming in a continuous loop.

18. The method of claim 17, wherein the repeating results in vibration of the piezoelectric patch.

19. The method of claim 16, further comprising selecting a desired sound level and/or spectral character manually by a motor vehicle user.

20. The method of claim 16, further comprising setting a desired sound level and/or spectral character automatically based on a time and/or location of a motor vehicle.
